SEQUENCE LISTING (16 sequences)

SEQ ID NO: 1 - DNA, 19 bp, Artificial Sequence
  tetracycline operator (tetO) sequence
tccctatcagtgatagaga

SEQ ID NO: 2 - DNA, 15 bp, Artificial Sequence
  tetracycline operator (tetO) sequence
cctatcagtgataga

SEQ ID NO: 3 - DNA, 15 bp, Artificial Sequence
  tetracycline operator (tetO) sequence
cctgtcagtgacaga

SEQ ID NO: 4 - DNA, 15 bp, Artificial Sequence
  tetracycline operator (tetO) sequence
cccatcagtgatgga

SEQ ID NO: 5 - DNA, 15 bp, Artificial Sequence
  tetracycline operator (tetO) sequence
cccgtcagtgacgga

SEQ ID NO: 6 - DNA, 15 bp, Artificial Sequence
  tetracycline operator (tetO) sequence
cctatcagtgacgga

SEQ ID NO: 7 - DNA, 747 bp, Artificial Sequence
  tetracycline controlled transactivator encoding sequence
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 8 - protein, 248 aa, Artificial Sequence
  tetracycline controlled transactivator sequence
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 9 - DNA, 751 bp, Artificial Sequence
  tetracycline controlled transactivator encoding sequence
atgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactag

SEQ ID NO: 10 - protein, 248 aa, Artificial Sequence
  tetracycline controlled transactivator sequence
MSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 11 - DNA, 747 bp, Artificial Sequence
  tetracycline controlled transactivator encoding sequence
atgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 12 - protein, 248 aa, Artificial Sequence
  tetracycline controlled transactivator sequence
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 13 - DNA, 747 bp, Artificial Sequence
  tetracycline controlled transactivator encoding sequence
atgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 14 - protein, 248 aa, Artificial Sequence
  tetracycline controlled transactivator sequence
MSRLDKSKVINGALELLNGVGIEGLTTRKLAQKLGVEQPTLYWHVKNKRALLDALPIEMLDRHHTHFCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPTDALDDFDLDMLPADALDDFDLDMLPADALDDFDLDMLPG

SEQ ID NO: 15 - DNA, 1008 bp, Artificial Sequence
  tetracycline controlled transactivator encoding sequence
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 16 - protein, 335 aa, Artificial Sequence
  tetracycline controlled transactivator sequence
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

(19) United States
(12) Patent Application Publication
(10) Pub. No.: US 2019/0161759 A1
(43) Pub. Date: May 30, 2019

(54) AMPLIFIED PRODUCTION OF MONOCLONAL ANTIBODIES

(71) Applicant: UNIVERSITY OF CINCINNATI, Cincinnati, OH (US)

(72) Inventors: Christian I. Hong, Cincinnati, OH (US); Toru Matsuura, Hirakata (JP); Kaoru Matsuura, Osaka (JP)

(21) Appl. No.: 16/203,148

(22) Filed: Nov. 28, 2018

Related U.S. Application Data
(60) Provisional application No. 62/591,328, filed on Nov. 28, 2017.

(51) Int. Cl. C12N 15/62; C07K 16/46

(52) U.S. Cl. CPC C12N 15/625; C07K 16/46; C12N 2830/003; C07K 2319/30; C12N 2840/002; C07K 2317/51

(57) ABSTRACT

Compositions and methods for production of monoclonal antibodies are provided according to aspects of the present invention including a synthetic gene circuit system, comprising: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, functioning of the system includes a positive feedback loop which amplifies expression of the heavy chain component of the monoclonal antibody and the light chain component of the monoclonal antibody.

### REFERENCE TO RELATED APPLICATION

This application claims priority to U.S. Provisional Patent Application Ser. No. 62/591,328, filed Nov. 28, 2017, the entire content of which is incorporated herein by reference.

### FIELD OF THE INVENTION

The present invention relates generally to production of monoclonal antibodies. According to specific aspects, the present invention relates to compositions providing a synthetic, positive feedback loop, amplifying production of monoclonal antibodies (MAB).

### BACKGROUND OF THE INVENTION

In the last 30 years, antibody-based immunotherapies have been applied to treat a variety of diseases. Monoclonal antibodies now represent over 30% of biopharmaceuticals in clinical trials. There is a continuing need for compositions and methods for preparation of voluminous quantities of MAB for clinical and other applications.

### SUMMARY OF THE INVENTION

Synthetic gene circuit systems are provided according to aspects of the present invention which include: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody.

According to aspects of the present invention, the light chain component of the monoclonal antibody is a human light chain and the heavy chain component of the monoclonal antibody is a human heavy chain.

According to aspects of the present invention, the light chain component of the monoclonal antibody is a human kappa light chain or human lambda light chain.

According to aspects of the present invention, the heavy chain component of the monoclonal antibody is selected from the group consisting of: human gamma heavy chain (IgG), human gamma heavy chain (IgM), human gamma heavy chain (IgD), human gamma heavy chain (IgA), and human gamma heavy chain (IgE).

According to aspects of the present invention, the light chain component of the monoclonal antibody is a human light chain, humanized light chain, chimeric light chain, or a combination of any two or more thereof; and the heavy chain component of the monoclonal antibody is a human heavy chain, humanized heavy chain, chimeric heavy chain, or a combination of any two or more thereof.

According to aspects of the present invention, the light chain component of the monoclonal antibody is a fragment of a human light chain, humanized light chain, chimeric light chain, or a combination of any two or more thereof; and the heavy chain component of the monoclonal antibody is a fragment of a human heavy chain, humanized heavy chain, chimeric heavy chain, or a combination of any two or more thereof.

According to aspects of the present invention, the light chain component of the monoclonal antibody is a human light chain, humanized light chain, chimeric light chain, or a combination of any two or more thereof; and the heavy chain component of the monoclonal antibody is a fragment of a human heavy chain, humanized heavy chain, chimeric heavy chain, or a combination of any two or more thereof.

Synthetic gene circuit systems are provided according to aspects of the present invention which include: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein one or more of the expression constructs is incorporated in a vector, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody.

Synthetic gene circuit systems are provided according to aspects of the present invention which include: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein one or more of the expression constructs is incorporated in a vector selected from the group consisting of: plasmid, adenovirus, adeno-associated virus, retrovirus, and lentivirus, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody.

According to aspects of the present invention, the constitutive promoter is cytomegalovirus (CMV) promoter.

Host cells including a synthetic gene circuit system are provided according to aspects of the present invention wherein the synthetic gene circuit system includes: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody. According to aspects of the present invention, the host cells are mammalian cells.

Methods of producing a monoclonal antibody are provided according to aspects of the present invention which include collecting the monoclonal antibody expressed by the synthetic gene circuit system present in a host cell wherein the synthetic gene circuit system includes: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody.

Methods of producing a monoclonal antibody are provided according to aspects of the present invention which include collecting and purifying the monoclonal antibody expressed by the synthetic gene circuit system present in a host cell wherein the synthetic gene circuit system includes: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody.

Methods of producing a monoclonal antibody are provided according to aspects of the present invention which include introducing a synthetic gene circuit system into a host cell, wherein the synthetic gene circuit system includes: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody; and collecting the monoclonal antibody expressed by the synthetic gene circuit system.

Methods of producing a monoclonal antibody are provided according to aspects of the present invention which include introducing a synthetic gene circuit system into a host cell, wherein the synthetic gene circuit system includes: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody; and collecting the monoclonal antibody expressed by the synthetic gene circuit system; and purifying the monoclonal antibody.

An expression construct is provided according to aspects of the present invention which includes a nucleic acid encoding tetracycline transactivator (tTA) operably linked to a tetracycline responsive element.

Kits for producing a monoclonal antibody are provided according to aspects of the present invention which include a synthetic gene circuit system including: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody.

Kits for producing a monoclonal antibody are provided according to aspects of the present invention which include a synthetic gene circuit system including: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody; and a host cell.

Kits for producing a monoclonal antibody are provided according to aspects of the present invention which include a host cell including a synthetic gene circuit system, wherein the synthetic gene circuit system includes: a first expression construct comprising a nucleic acid encoding tetracycline transactivator (tTA) operably linked to a ubiquitous promoter; a second expression construct comprising a nucleic acid encoding tetracycline transactivator (tTA) operably linked to a tetracycline responsive element (TRE); a third expression construct comprising a nucleic acid encoding a heavy chain component of an monoclonal antibody operably linked to a TRE; and a fourth expression construct comprising a nucleic acid encoding a light chain component of an monoclonal antibody operably linked to a TRE, wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody. According to aspects of the present invention, the host cells are mammalian cells.

### BRIEF DESCRIPTION OF THE DRAWINGS

FIG. 1A is a diagram of an expression vector including: a nucleic acid encoding tetracycline transactivator (tTA) operably linked to a tetracycline responsive element (TRE);

FIG. 1B is a diagram of an expression vector including: a nucleic acid encoding tetracycline transactivator (tTA) operably linked to a cytomegalovirus (CMV) ubiquitous promoter;

FIG. 1C is a diagram of an expression vector including: a nucleic acid encoding a heavy chain of an IgG monoclonal antibody operably linked to a TRE;

FIG. 1D is a diagram of an expression vector including: a nucleic acid encoding a light chain of an IgG monoclonal antibody operably linked to a TRE;

FIG. 2 is a diagram illustrating a positive feedback loop involving production of tTA under the control of a ubiquitous promoter (CMV) from a first expression construct and activity of the tTA on the TRE of three additional expression constructs, amplifying production of the heavy chain and light chain of an IgG monoclonal antibody;

FIG. 3A is a graph showing increased mRNA production of the light chain of a monoclonal antibody according to a method of the present invention compared to a control on Day 4;

FIG. 3B is a graph showing increased mRNA production of the heavy chain of a monoclonal antibody according to a method of the present invention compared to a control on Day 4; and

FIG. 4 is a graph showing increased production of a monoclonal antibody according to a method of the present invention compared to a control.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; and Herdewijn, P. (Ed.), Oligonucleotide Synthesis: Methods and Applications, Methods in Molecular Biology, Humana Press, 2004.

The singular terms “a,” “an,” and “the” are not intended to be limiting and include plural referents unless explicitly stated otherwise or the context clearly indicates otherwise.

A synthetic gene circuit system is provided according to aspects of the present invention which includes: 1) a first expression construct comprising a nucleic acid encoding tetracycline transactivator (tTA) operably linked to a constitutive promoter; 2) a second expression construct comprising a nucleic acid encoding tTA operably linked to a tetracycline responsive element (TRE); 3) a third expression construct comprising a nucleic acid encoding a heavy chain component of an monoclonal antibody operably linked to a TRE; and 4) a fourth expression construct comprising a nucleic acid encoding a light chain component of an monoclonal antibody operably linked to a TRE, wherein expression of tTA by the first expression construct stimulates expression of tTA by the second expression construct; wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody.

The term “synthetic gene circuit” as used herein refers to a plurality of non-naturally occurring expression constructs which are functionally linked so that an expression product of at least one of the expression constructs affects or controls the levels of an expression product of at least one other expression construct.

The term “nucleic acid” refers to RNA or DNA molecules having more than one nucleotide in any form including single-stranded, double-stranded, oligonucleotide or polynucleotide. The term “nucleotide sequence” refers to the ordering of nucleotides in an oligonucleotide or polynucleotide and is usually shown as the ordering of the sense strand.

The term “constitutive promoter” refers to promoter sequences that are unregulated in vivo and hence allow for continual transcription of an operably linked nucleic acid, such as a nucleic acid encoding tTA. Constitutive promoters useful in mammalian systems include, but are not limited to, cytomegalovirus immediate-early promoter (CMV), simian virus 40 early promoter (SV40), rous sarcoma virus (RSV) promoter, human elongation factor 1a promoter (EF1A), human ubiquitin C promoter (UBC), mouse phospholycerate kinase 1 promoter (PGK), and chicken b-actin promoter. Such promoter sequences can be obtained commercially, isolated from naturally occurring sources, isolated from existing expression constructs, or synthesized by recombinant or chemical synthetic techniques.

A tetracycline transactivator (also called tetracycline-controlled transactivator and tetracycline-regulated transactivator, all abbreviated tTA) is a fusion protein including the Tet repressor DNA binding protein (TetR) from the Tc resistance operon of E. coli transposon Tn10 fused to the strong transactivating C-terminal domain of virion protein 16 (VP16) from Herpes simplex virus (HSV), see for example, Suhr et al., J. Cell Biol., 153(2):283-294, 2001; Park et al., Eukaryotic Cell, 4(8): 1328-1342, 2005; and Gossen et al., Proc. Natl. Acad. Sci. USA, 89:5547-5551, 1992. A rtTA (reverse tetracycline-controlled transactivator) can be used in a “Tet On” configuration where stimulation of expression in the presence of tetracycline or an analog, such as doxycycline, is desired. Example, non-limiting, nucleic acid sequences encoding tetracycline-controlled transactivators or reverse tetracycline-controlled transactivators are shown herein along with the respective encoded tTA and rtTA proteins, see SEQ ID NOs: 7, 8, 9, 10, 11, 12, 13, 14, 15, and 16.

A TRE includes a tetracycline operator (tetO) sequence to which tTA binds in the absence of tetracycline or an analog, such as doxycycline, (“Tet Off”). Binding of tTA to the TRE activates transcription of an operably linked nucleic acid sequence. Alternatively, a “Tet On” configuration can be used in which rtTA binds a TRE in the presence of tetracycline or doxycycline.

A TRE may include two or more repeats of a tetracycline operator (tetO) sequence such as 2, 3, 4, 5, 6, 7, 8, 9, 10, or more repeats of the tetO sequence, and is recognized by the tetracycline repressor (tetR). Where more than one tetO sequence is present, the tetO sequences may be contiguous, a spacer can be included between the tetO sequences, or some tetO sequences may be contiguous and some may have a spacer interposed between them.

A TRE may include two or more repeats of a tetracycline operator (tetO) sequence TCCCTATCAGTGATAGAGA, SEQ ID NO:1, such as 2, 3, 4, 5, 6, 7, 8, 9, 10, or more repeats of the tetO sequence TCCCTATCAGTGATAGAGA, SEQ ID NO:1, and is recognized by the tetracycline repressor (tetR).

Additional tetO sequences are known and can be used in expression constructs according to aspects of the present invention, such as, but not limited to, CCTATCAGTGATAGA (SEQ ID NO:2), CCTGTCAGTGACAGA (SEQ ID NO:3), CCCATCAGTGATGGA (SEQ ID NO:4), CCCGTCAGTGACGGA (SEQ ID NO: 5), and CCTATCAGTGACGGA (SEQ ID NO:6).

A TRE may include two or more repeats of a tetO such as, but not limited to, SEQ ID NO:1, SEQ ID NO:2, SEQ ID NO:3, SEQ ID NO:4, SEQ ID NO:5, or SEQ ID NO:6, such as 2, 3, 4, 5, 6, 7, 8, 9, 10, or more repeats of one or more tetO sequences, and is recognized by the tetracycline repressor (tetR).

The structural basis of tetracycline repressor interaction with tetracycline operator is well established, see for example, Orth et al., 2000, Nature Structural Biology, 7(3):215-219.

The phrase “amplifying expression” as used herein refers to increasing the number of monoclonal antibody heavy chains and light chains produced compared to a conventional monoclonal antibody production method which does not include a positive feedback loop in a synthetic gene circuit.

The term “positive feedback loop” as used herein refers to a system where expression of tTA increases the expression of tTA via binding to a TRE which is in operable linkage to a nucleic acid encoding tTA in an expression construct.

The term “expression construct” is used herein to refer to a double-stranded recombinant DNA molecule containing a desired nucleic acid coding sequence for a protein to be expressed and containing one or more regulatory elements necessary or desirable for the expression of the operably linked coding sequence. The terms “expressed” and “expression” refer to transcription of a nucleic acid sequence to produce a corresponding mRNA and/or translation of the mRNA to produce the corresponding protein. Expression constructs can be generated recombinantly or by DNA synthesis using well-known methodology.

The term “recombinant” is used to indicate a nucleic acid construct in which two or more nucleic acids are linked and which are not found linked in nature.

The term “regulatory element” as used herein refers to a nucleotide sequence which controls some aspect of the expression of nucleic acid sequences. Exemplary regulatory elements illustratively include an enhancer, an internal ribosome entry site (IRES), an intron; an origin of replication, a polyadenylation signal (polyA), a promoter, a transcription termination sequence, and an upstream regulatory domain, which contribute to the replication, transcription, post-transcriptional processing of a nucleic acid sequence. Those of ordinary skill in the art are capable of selecting and using these and other regulatory elements in an expression construct with no more than routine experimentation.

Expression constructs operable to express a desired protein include, for example, in operable linkage: a promoter, a DNA sequence encoding a desired protein and a transcription termination site.

Expression constructs can be generated recombinantly or synthetically using well-known methodology.

The term “operably linked” as used herein refers to a nucleic acid in functional relationship with a second nucleic acid.

A regulatory element included in an expression construct is a promoter in particular aspects.

The term “promoter” is well-known in the art and refers to one or more DNA sequences operably linked to a nucleic acid sequence to be transcribed and which bind an RNA polymerase and allow for initiation of transcription. A promoter is typically positioned upstream (5′) of a nucleic acid encoding a peptide or protein to be expressed.

An mRNA polyadenylation (pA) sequence may be included such as, but not limited to SV40-pA, beta-globin-pA and SCF-pA.

An expression construct may include sequences necessary for amplification in bacterial cells, such as a selection marker (e.g. kanamycin or ampicillin resistance gene) and a replicon.

An internal ribosome entry site (IRES) is an optionally included nucleic acid sequence that permits translation initiation at an internal site in an mRNA. IRES are well-known in the art, for example as described in Pelletier, J. et al., Nature, 334:320-325, 1988; Vagner, S. et al., EMBO Rep., 2:893-898, 2001; and Hellen, C. U. et al, Genes Dev. 15:1593-1612, 2001.

The term “transcription termination site” refers to a DNA sequence operable to terminate transcription by an RNA polymerase. A transcription termination site is generally positioned downstream (3′) of a nucleic acid encoding a peptide or protein to be expressed.

A leader sequence is optionally included in an expression construct.

An expression construct can be cloned into an expression vector for transformation into prokaryotic or eukaryotic cells and expression of the encoded peptides and/or protein(s). As used herein, “expression vectors” are defined as polynucleotides which, when introduced into an appropriate host cell or in a cell-free expression system, can be transcribed and translated, producing the encoded polypeptide(s).

Expression vectors are known in the art and include plasmids, cosmids, viruses and bacteriophages, for example. Expression vectors can be prokaryotic vectors, insect vectors, or eukaryotic vectors, for example.

For example, an expression construct including, in operable linkage: a promoter, a DNA sequence encoding a desired protein and a transcription termination site, is included in a plasmid, cosmid, BAC, YAC, virus or bacteriophage expression vector. Particular viral vectors illustratively include those derived from adenovirus, adeno-associated virus and lentivirus.

Particular vectors are known in the art and one of skill in the art will recognize an appropriate vector for a specific purpose.

Any suitable expression vector/host cell system can be used for expression according to aspects of the present invention.

Expression of a desired protein using a recombinant expression vector is accomplished according to aspects of the present invention by introduction of the expression vector into a eukaryotic or prokaryotic host cell expression system such as an insect cell, mammalian cell, yeast cell, fungus, bird egg, bacterial cell or any other single or multicellular organism recognized in the art.

Host cells containing the recombinant expression vector are maintained under conditions wherein the desired protein is produced. Host cells may be cultured and maintained using known cell culture techniques such as described in Celis, Julio, ed., 1994, Cell Biology Laboratory Handbook, Academic Press, N.Y. Various culturing conditions for these cells, including media formulations with regard to specific nutrients, oxygen, tension, carbon dioxide and reduced serum levels, can be selected and optimized by one of skill in the art.

For expression in a host cell, any of the well-known procedures for introducing recombinant nucleic acids into host cells may be used, such as calcium phosphate transfection, polybrene, protoplast fusion, electroporation, sonoporation, liposomes and microinjection, examples of which are described in Sambrook et al., Molecular Cloning: A Laboratory Manual, Cold Spring Harbor Laboratory Press, 2001; and Ausubel, F. et al., (Eds.), Current Protocols in Molecular Biology, 2014.

According to aspects of the present invention, a host cell including a synthetic gene circuit system is provided according to aspects of the present invention wherein the host cell includes: 1) a first expression construct comprising a nucleic acid encoding tetracycline transactivator (tTA) operably linked to a constitutive promoter; 2) a second expression construct comprising a nucleic acid encoding tTA operably linked to a tetracycline responsive element (TRE); 3) a third expression construct comprising a nucleic acid encoding a heavy chain component of an monoclonal antibody operably linked to a TRE; and 4) a fourth expression construct comprising a nucleic acid encoding a light chain component of an monoclonal antibody operably linked to a TRE, wherein expression of tTA by the first expression construct stimulates expression of tTA by the second expression construct; wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody.

The host cell can be any host cell compatible with expression activity of tTA and production of the heavy chain component of the monoclonal antibody and the light chain component of the monoclonal antibody.

According to aspects of the present invention, the host cell is a mammalian cell.

According to aspects of the present invention, the host cell is a cell line cell such as, but not limited to, a Chinese hamster ovary cell (CHO), a human embryonic kidney 293 (HEK293) cell, baby hamster kidney cell (BHK), monkey kidney cell (CV-1), African green monkey kidney cell (VERO), murine myeloma cell (NS0 or Sp2/0), human liver cell (Hep G2), human lung cell (W138), human cervical carcinoma cell (HELA), mouse sertoli cell (TM4), or canine kidney cell (MDCK).

A cell-free expression system is optionally used to express a desired, such as described in Ausubel, F. et al., (Eds.), Current Protocols in Molecular Biology, 2014.

tTAs, rtTAs, TREs, and variants of any thereof, can be used in methods according to aspects described herein.

As used herein, the term “variant” refers to a variation of a nucleic acid sequence, a variation of a nucleic acid sequence encoding a protein, or a variation of a protein in which one or more nucleotides or amino acid residues have been modified by nucleotide or amino acid substitution, addition, or deletion while retaining the function of the reference nucleic acid sequence or protein. Variants of a nucleic acid sequence or protein described herein are characterized by conserved functional properties compared to the corresponding nucleic acid sequence or protein.

Mutations can be introduced using standard molecular biology techniques, such as chemical synthesis, site-directed mutagenesis and PCR-mediated mutagenesis.

One of skill in the art will recognize that one or more amino acid mutations can be introduced without altering the functional properties of a desired protein. For example, one or more amino acid substitutions, additions, or deletions can be made without altering the functional properties of a desired protein.

Biological activity of a protein variant is readily determined by one of skill in the art, for instance using any of the functional assays described herein or other functional assays known in the art.

Variants of a protein described herein are characterized by conserved functional properties compared to the corresponding protein and have 75%, 80%, 85%, 90%, 95%, 96%, 97%, 98%, 99% or greater identity to the amino acid sequence of a reference protein.

When comparing a reference protein to a variant, amino acid similarity may be considered in addition to identity of amino acids at corresponding positions in an amino acid sequence. “Amino acid similarity” refers to amino acid identity and conservative amino acid substitutions in a putative homologue compared to the corresponding amino acid positions in a reference protein.

Variants of a protein described herein are characterized by conserved functional properties compared to the corresponding protein and have 75%, 80%, 85%, 90%, 95%, 96%, 97%, 98%, 99% or greater similarity to the amino acid sequence of a reference protein.

Conservative amino acid substitutions can be made in reference proteins to produce variants.

Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

A variant can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

Percent identity is determined by comparison of amino acid or nucleic acid sequences, including a reference amino acid or nucleic acid sequence and a putative homologue amino acid or nucleic acid sequence. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). The two sequences compared are generally the same length or nearly the same length.

The determination of percent identity between two sequences can also be accomplished using a mathematical algorithm. Algorithms used for determination of percent identity illustratively include the algorithms of S. Karlin and S. Altshul, PNAS, 90:5873-5877, 1993; T. Smith and M. Waterman, Adv. Appl. Math. 2:482-489, 1981, S. Needleman and C. Wunsch, J. Mol. Biol., 48:443-453, 1970, W. Pearson and D. Lipman, PNAS, 85:2444-2448, 1988 and others incorporated into computerized implementations such as, but not limited to, GAP, BESTFIT, FASTA, TFASTA; and BLAST, for example incorporated in the Wisconsin Genetics Software Package, Genetics Computer Group, 575 Science Drive, Madison, Wis.) and publicly available from the National Center for Biotechnology Information.

A non-limiting example of a mathematical algorithm utilized for the comparison of two sequences is the algorithm of Karlin and Altschul, 1990, PNAS 87:2264-2268, modified as in Karlin and Altschul, 1993, PNAS. 90:5873-5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, word length=12 to obtain nucleotide sequences homologous to a nucleic acid molecules of the present invention. BLAST protein searches are performed with the XBLAST program parameters set, e.g., to score 50, word length=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389-3402. Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

One of skill in the art will recognize that one or more nucleic acid or amino acid mutations can be introduced without altering the functional properties of a given nucleic acid or protein, respectively.

Furthermore, it is appreciated that due to the degenerate nature of the genetic code, alternate nucleic acid sequences encode a specified protein, and that such alternate nucleic acids may be expressed to produce the desired protein.

The term “monoclonal antibody” as used herein refers to a substantially homogeneous population of antibodies produced by expression of the light chain component and heavy chain component included in expression constructs which are included in a synthetic gene circuit according to the present invention. The term “monoclonal antibody” refers to the characteristic of the antibody as being obtained by production of a substantially homogeneous population of antibodies according to aspects of the present invention and does not implicate a requirement that the antibody be produced by another method such as a traditional hybridoma method, phage display method, or transgenic animal method. As will be recognized by the skilled artisan, spontaneous mutations may occasionally occur during production such that the population of antibodies produced by expression of the light chain component and heavy chain component included in expression constructs which are included in a synthetic gene circuit according to the present invention is not entirely homogeneous, but such variants are minor in the amount present in the population.

Monoclonal antibodies and antigen-binding fragments thereof are known in the art, for instance, as described in Antibody Engineering, Kontermann, R. and Dübel, S. (Eds.), Springer, 2001; Harlow, E. and Lane, D., Antibodies: A Laboratory Manual, Cold Spring Harbor Laboratory Press, 1988; Ausubel, F. et al., (Eds.), Short Protocols in Molecular Biology, Wiley, 2002; J. D. Pound (Ed.) Immunochemical Protocols, Methods in Molecular Biology, Humana Press, 2nd ed., 1998; B. K. C. Lo (Ed.), Antibody Engineering: Methods and Protocols, Methods in Molecular Biology, Humana Press, 2003; and Kohler, G. and Milstein, C., Nature, 256:495-497 (1975).

Generally described, “intact” monoclonal antibodies contain two identical heavy chain polypeptides and two identical light chain polypeptides. Antigen recognition is mediated by variable regions of the heavy and light chains. Complementarity determining region (CDR) refers to polypeptide regions within the variable region of heavy and light chains. Three CDRs (CDR1, CDR2 and CDR3) are present in each light chain variable region (V_{L}) and each heavy chain variable region (V_{H}). The CDRs are generally responsible for specific antigen recognition properties of the antibody or antigen-binding fragment.

The monoclonal antibodies produced by a method according to aspects of the present invention can be intact antibodies or antibody fragments of various types, including, but not limited to Fab, Fab′, F(ab′)2, Fv, and diabodies.

The monoclonal antibodies produced by a method according to aspects of the present invention can be any of various classes including IgG, IgM, IgA, IgE and IgD.

According to aspects of the present invention, the light chain component of the monoclonal antibody is a human light chain and the heavy chain component of the monoclonal antibody is a human heavy chain.

According to aspects of the present invention, the light chain component of the monoclonal antibody is a human kappa light chain or human lambda light chain.

According to aspects of the present invention, the heavy chain component of the monoclonal antibody is selected from the group consisting of: human gamma heavy chain (IgG), human gamma heavy chain (IgM), human gamma heavy chain (IgD), human gamma heavy chain (IgA), and human gamma heavy chain (IgE).

According to aspects of the present invention, the light chain component of the monoclonal antibody is a human light chain, humanized light chain, chimeric light chain, or a combination of any two or more thereof; and the heavy chain component of the monoclonal antibody is a human heavy chain, humanized heavy chain, chimeric heavy chain, or a combination of any two or more thereof.

According to aspects of the present invention, the light chain component of the monoclonal antibody is a fragment of a human light chain, humanized light chain, chimeric light chain, or a combination of any two or more thereof; and the heavy chain component of the monoclonal antibody is a fragment of a human heavy chain, humanized heavy chain, chimeric heavy chain, or a combination of any two or more thereof.

According to aspects of the present invention, the light chain component of the monoclonal antibody is a human light chain, humanized light chain, chimeric light chain, or a combination of any two or more thereof; and the heavy chain component of the monoclonal antibody is a fragment of a human heavy chain, humanized heavy chain, chimeric heavy chain, or a combination of any two or more thereof.

The term “human antibody,” “human light chain,” and “human heavy chain” refers to proteins having variable and constant regions derived from human germline immunoglobulin sequences. Human antibodies, human light chains and human heavy chains may include amino acid sequence differences compared to human germline immunoglobulin sequences due to spontaneous or engineered mutations.

The term “chimeric” refers to a monoclonal antibody, heavy chain thereof, and/or light chain thereof, in which at least a portion of the heavy and/or light chain is derived from a particular source or species and the remainder of the heavy and/or light chain is derived from at least one different source or species.

The term “humanized” refers to a monoclonal antibody, heavy chain thereof, and/or light chain thereof, having an antigen binding site that is derived from an immunoglobulin of a non-human species and the remaining portion of the antibody, heavy chain thereof, and/or light chain thereof, is derived from a human, i.e. is based on the structure and/or amino acid sequence of a human immunoglobulin.

Methods of producing a monoclonal antibody are provided according to aspects of the present invention which include collecting the monoclonal antibody expressed by a synthetic gene circuit system which includes: 1) a first expression construct comprising a nucleic acid encoding tetracycline transactivator (tTA) operably linked to a constitutive promoter; 2) a second expression construct comprising a nucleic acid encoding tTA operably linked to a tetracycline responsive element (TRE); 3) a third expression construct comprising a nucleic acid encoding a heavy chain component of an monoclonal antibody operably linked to a TRE; and 4) a fourth expression construct comprising a nucleic acid encoding a light chain component of an monoclonal antibody operably linked to a TRE, wherein expression of tTA by the first expression construct stimulates expression of tTA by the second expression construct; wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody.

According to aspects of the present invention, the method of producing the monoclonal antibody includes introducing a synthetic gene circuit system which includes: 1) a first expression construct comprising a nucleic acid encoding tetracycline transactivator (tTA) operably linked to a constitutive promoter; 2) a second expression construct comprising a nucleic acid encoding tTA operably linked to a tetracycline responsive element (TRE); 3) a third expression construct comprising a nucleic acid encoding a heavy chain component of an monoclonal antibody operably linked to a TRE; and 4) a fourth expression construct comprising a nucleic acid encoding a light chain component of an monoclonal antibody operably linked to a TRE, into a host cell; and collecting the monoclonal antibody expressed by the system. As described above, for expression in a host cell, any of the well-known procedures for introducing recombinant nucleic acids into host cells may be used, such as calcium phosphate transfection, polybrene, protoplast fusion, electroporation, sonoporation, liposomes and microinjection, examples of which are described in Sambrook et al., Molecular Cloning: A Laboratory Manual, Cold Spring Harbor Laboratory Press, 2001; and Ausubel, F. et al., (Eds.), Current Protocols in Molecular Biology, 2014.

According to aspects of the present invention, the method of producing the monoclonal antibody includes purifying the monoclonal antibody to produce a purified monoclonal antibody.

The term “purified” as used herein refers to separation of a monoclonal antibody from at least one other component of the cell or cell-free system in which it was produced.

According to aspects of the present invention, the monoclonal antibody is purified such that it is substantially free of other proteins.

Monoclonal antibody purification is achieved by techniques illustratively including electrophoretic methods such as gel electrophoresis and 2-D gel electrophoresis; chromatography methods such as HPLC, ion exchange chromatography, affinity chromatography, size exclusion chromatography, thin layer and paper chromatography.

Monoclonal antibodies that can be produced using methods and compositions according to the present invention include, but are not limited to, 3F8, 8H9, abagovomab, abituzumab, abrilumab, actoxumab, adalimumab, adecatumumab, aducanumab, afasevikumab, afutuzumab, alemtuzumab, alirocumab, altumomab, amatuximab, anetumab, anifrolumab, anrukinzumab, apolizumab, aprutumab, arcitumomab, ascrinvacumab, aselizumab, atezolizumab, atidortoxumab, atinumab, atorolimumab, avelumab, azintuxizumab, bapineuzumab, basiliximab, bavituximab, begelomab, belimumab, benralizumab, bermekimab, bertilimumab, besilesomab, bevacizumab, bezlotoxumab, bimagrumab, bimekizumab, bivatuzumab, bleselumab, blontuvetmab, blosozumab, bococizumab, brazikumab, brentuximab, briakinumab, brodalumab, brontictuzumab, burosumab, cabiralizumab, camidanlumab, camrelizumab, canakinumab, cantuzumab, cantuzumab, capromab, carlumab, carotuximab, catumaxomab, cedelizumab, cemiplimab, cergutuzumab, cetuximab, cixutumumab, clazakizumab, clenoliximab, clivatuzumab, codrituzumab, coltuximab, conatumumab, concizumab, crenezumab, crotedumab, dacetuzumab, daclizumab, dalotuzumab, dapirolizumab, daratumumab, dectrekumab, demcizumab, denintuzumab, denosumab, depatuxizumab, derlotuximab, detumomab, dinutuximab, diridavumab, domagrozumab, drozitumab, duligotuzumab, dupilumab, durvalumab, dusigitumab, ecromeximab, eculizumab, edobacomab, edrecolomab, efalizumab, eldelumab, elgemtumab, elotuzumab, elsilimomab, emactuzumab, emapalumab, emibetuzumab, emicizumab, enoblituzumab, enfortumab, enavatuzumab, enfortumab, enlimomab, enoblituzumab, enokizumab, enoticumab, ensituximab, epitumomab, epratuzumab, eptinezumab, erenumab, ertumaxomab, etaracizumab, etrolizumab, evinacumab, evolocumab, exbivirumab, fanolesomab, faralimomab, farletuzumab, fasinumab, felvizumab, fezakinumab, ficlatuzumab, figitumumab, firivumab, flanvotumab, fletikumab, fontolizumab, foralumab, foravirumab, fremanezumab, fresolimumab, fulranumab, futuximab, galcanezumab, galiximab, ganitumab, gantenerumab, gavilimomab, gemtuzumab, gevokizumab, girentuximab, glembatumumab, golimumab, gomiliximab, guselkumab, ianalumab, ibalizumab, ibritumomab, icrucumab, idarucizumab, ifabotuzumab, igovomab, imab362, imalumab, imciromab, imgatuzumab, inclacumab, indatuximab, indusatumab, inebilizumab, infliximab, inolimomab, inotuzumab, intetumumab, ipilimumab, iratumumab, isatuximab, istiratumab, itolizumab, ixekizumab, keliximab, labetuzumab, ladiratuzumab, lanadelumab, landogrozumab, laprituximab, larcaviximab, lebrikizumab, lemalesomab, lenzilumab, lerdelimumab, lexatumumab, lifastuzumab, ligelizumab, lilotomab, lintuzumab, lirilumab, lokivetmab, loncastuximab, lorvotuzumab, lucatumumab, lulizumab, lumiliximab, lumretuzumab, lupartumab, lutikizumab, mapatumumab, margetuximab, matuzumab, mavrilimumab, mepolizumab, metelimumab, milatuzumab, minretumomab, mirikizumab, mirvetuximab, mitumomab, mogamulizumab, monalizumab, morolimumab, mosunetuzumab, motavizumab, moxetumomab, muromonab-CD3, namilumab, naptumomab, naratuximab, namatumab, natalizumab, necitumumab, nemolizumab, nerelimomab, nesvacumab, nimotuzumab, nivolumab, obiltoxaximab, obinutuzumab, ocaratuzumab, ocrelizumab, odulimomab, ofatumumab, olaratumab, oleclumab, olokizumab, omalizumab, omburtamab, onartuzumab, ontuxizumab, opicinumab, oregovomab, orticumab, otelixizumab, otlertuzumab, oxelumab, ozanezumab, pagibaximab, palivizumab, pamrevlumab, panitumumab, pankomab, panobacumab, parsatuzumab, pascolizumab, pateclizumab, patritumab, pembrolizumab, pemtumomab, perakizumab, pertuzumab, pidilizumab, pinatuzumab, placulumab, pidilizumab, plozalizumab, polatuzumab, ponezumab, prezalizumab, priliximab, pritoxaximab, pritumumab, quilizumab, racotumomab, radretumab, rafivirumab, ralpancizumab, ramucirumab, ranevetmab, ranibizumab, ravulizumab, raxibacumab, refanezumab, regavirumab, relatlimab, remtolumab, reslizumab, rilotumumab, rinucumab, risankizumab, rituximab, robatumumab, roledumab, romosozumab, rontalizumab, rovalpituzumab, rovelizumab, rozanolixizumab, ruplizumab, sacituzumab, samalizumab, sarilumab, satralizumab, satumomab, secukinumab, seribantumab, setoxaximab, sibrotuzumab, sifalimumab, siltuximab, simtuzumab, siplizumab, sirukumab, sofituzumab, solanezumab, sonepcizumab, sontuzumab, spartalizumab, stamulumab, sutimlimab, suvizumab, suvratoxumab, tabalumab, tacatuzumab, tadocizumab, talizumab, tanezumab, taplitumomab, tarextumab, tefibazumab, telimomab, telisotuzumab, tenatumomab, teneliximab, teplizumab, teprotumumab, tesidolumab, tetulomab, tezepelumab, theralizumab, tigatuzumab, tildrakizumab, timolumab, tislelizumab, tisotumab, tocilizumab, toralizumab, tositumomab, tovetumab, tralokinumab, trastuzumab, TRBS07, tregalizumab, tremelimumab, trevogrumab, tucotuzumab, tuvirumab, ublituximab, ulocuplumab, urelumab, ustekinumab, urtoxazumab, utomilumab, vadastuximab, vandortuzumab, vantictumab, vanucizumab, vapaliximab, varlilumab, vatelizumab, vedolizumab, veltuzumab, vesencumab, visilizumab, volociximab, vorsetuzumab, votumumab, xentuzumab, XMAB-5574, zalutumumab, zanolimumab, zatuximab, zenocutuzumab, ziralimumab, zolbetuximab, and zolimomab.

Kits are provided according to aspects of the present invention which include a synthetic gene circuit system which includes: 1) a first expression construct comprising a nucleic acid encoding tetracycline transactivator (tTA) operably linked to a constitutive promoter; 2) a second expression construct comprising a nucleic acid encoding tTA operably linked to a tetracycline responsive element (TRE); 3) a third expression construct comprising a nucleic acid encoding a heavy chain component of an monoclonal antibody operably linked to a TRE; and 4) a fourth expression construct comprising a nucleic acid encoding a light chain component of an monoclonal antibody operably linked to a TRE.

Kits are provided according to aspects of the present invention which include a synthetic gene circuit system which includes: 1) a first expression construct comprising a nucleic acid encoding tetracycline transactivator (tTA) operably linked to a constitutive promoter; 2) a second expression construct comprising a nucleic acid encoding tTA operably linked to a tetracycline responsive element (TRE); 3) a third expression construct comprising a nucleic acid encoding a heavy chain component of an monoclonal antibody operably linked to a TRE; and 4) a fourth expression construct comprising a nucleic acid encoding a light chain component of an monoclonal antibody operably linked to a TRE and at least one host cell.

Kits are provided according to aspects of the present invention which include a synthetic gene circuit system which includes: 1) a first expression construct comprising a nucleic acid encoding tetracycline transactivator (tTA) operably linked to a constitutive promoter; 2) a second expression construct comprising a nucleic acid encoding tTA operably linked to a tetracycline responsive element (TRE); 3) a third expression construct comprising a nucleic acid encoding a heavy chain component of an monoclonal antibody operably linked to a TRE; and 4) a fourth expression construct comprising a nucleic acid encoding a light chain component of an monoclonal antibody operably linked to a TRE and at least one host cell, wherein the synthetic gene circuit system is present in the host cell.

Optionally, one or more additional components are provided in a kit according to aspects of the present invention including, but not limited to, a host cell growth medium.

Embodiments of inventive compositions and methods are illustrated in the following examples. These examples are provided for illustrative purposes and are not considered limitations on the scope of inventive compositions and methods.

### EXAMPLES

A synthetic gene network with a positive feedback loop (PFL) that amplifies the expression of a monoclonal antibody, Trastuzumab, is described in this example which dramatically increases monoclonal antibody production (˜400% increased compared to the conventional system).

### Materials and Methods

Materials. pVITRO1-Trastuzumab-IgG3/κ was a gift from Dr. Andrew Beavil (King's College London, Addgene plasmid #61885). tet operator plasmid was a gift from Dr. Lynda Chin (University of Texas, Addgene plasmid #8901). pTetOff and pTRE2-hyg were purchased from Clontech.

Plasmid constructions. TRE-tTA was constructed with the insertion of tTA from pTetOff into the tet operator plasmid. TRE-IgG-HC was constructed with insertion of PCR amplified IgG-HC fragment from pVITRO1-Trastuzumab-IgG3/κ into pTRE2-hyg. TRE-IgG-LC was constructed with insertion of PCR amplified IgG-LC fragment from pVITRO1-Trastuzumab-IgG3/κ into the tet operator plasmid.

RNA extraction, Quantitative RT-PCR, and RNA sequencing. Total RNA was isolated using Tri Reagent (Molecular Research Center, Inc.), and treated with RQ1 RNase-free DNase (Promega). Complementary DNA (cDNA) was produced from the isolated RNA by using Reverse Transcription System from Promega. Quantitative RT-PCR (qRT-PCR) was performed with the cDNA by using StepOne plus system (Applied Biosystems).

Human IgG detection. Human IgG secreted into the cell culturing media was detected by Human IgG ELISA Kit (Sigma).

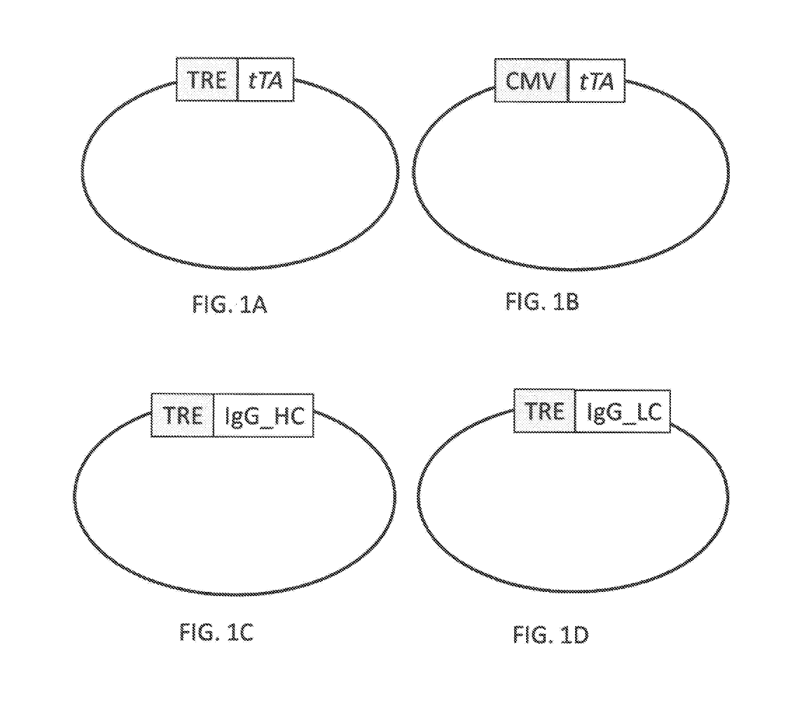

A plasmid with tetracycline transactivator (tTA) under the control of the tetracycline responsive element (TRE) was constructed. tTA can bind to the TRE promoter and activate its own transcription without the addition of tetracycline or doxycycline. Incorporated in the synthetic gene circuit of the present invention, this plasmid creates a synthetic gene network with a positive feedback loop to amplify the expression of tTA and any other target genes under the control of the TRE promoter (FIG. 1a). Based on the positive feedback loop of the tTA and TRE promoter, MAB production plasmid DNAs were developed and FIGS. 1a, 1b, 1c and 1d show plasmid DNAs used to amplify human IgG production in mammalian cells. FIG. 1a and FIG. 1b show that for the expression of the transcription factor tTA, two plasmid DNAs: (a) TRE-tTA and (b) CMV-tTA were generated and used. FIG. 1c and FIG. 1d show plasmid DNAs in which IgG-HC and IgG-LC are under the control of TRE promoter.

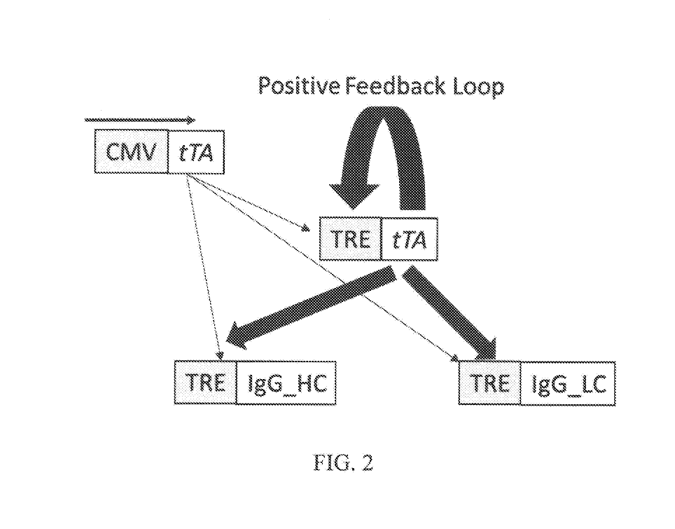

FIG. 2 shows a schematic diagram of the amplified system illustrating amplified production of human IgG HC and IgG LC and thus producing an IgG monoclonal antibody. FIG. 2 is a schematic diagram of the gene network to produce IgG HC and LC (arrows in FIG. 2). tTA transcription factor under the control of CMV promoter initially expresses and promotes the expression of factors under the control of TRE promoter. TRE-tTA expression is initiated by CMV-tTA, and expressed tTA promote expression of genes under the control of TRE promoter. Therefore, tTA can promote expression not only IgG-HC and LC but also own expression, which makes positive feedback loop to amplify the expression of tTA. The amplified tTA further overexpress IgG HC and LC.

In this example, the MAB production system contains four plasmid DNAs that incorporate: (i) CMV-tTA containing the CMV promoter which controls tTA expression to initiate the entire system; (ii) TRE-tTA to produce the positive feedback for overexpressing tTA; and (iii and iv) TRE-HC and TRE-LC to produce a MAB which is targeted to HER2 oncogene (Trastuzumab). Using these plasmid DNAs, mRNA expression level of Trastuzumab HC and Trastuzumab LC was compared with expression of pVITRO1-Trastuzumab-IgG3/κ a plasmid which contains HC and LC of Trastuzumab. In pVITRO1-Trastuzumab-IgG3/κ, HC and LC are under the control of EF1a promoter which is known as one of the strongest promoter in mammalian cells.

For this comparison, pVITRO1-Trastuzumab-IgG3/κ was transiently transfected into Human Embryonic Kidney (HEK) 293 cells. The synthetic gene circuit system including four plasmids (i) CMV-tTA; (ii) TRE-tTA; and (iii) TRE-Trastuzumab HC and TRE-Trastuzumab LC was transiently transfected into a parallel population of Human Embryonic Kidney (HEK) 293 cells. Transient transfections into HEK293 cells were performed with Lipofectamine 2000 (Lifetechnologies).

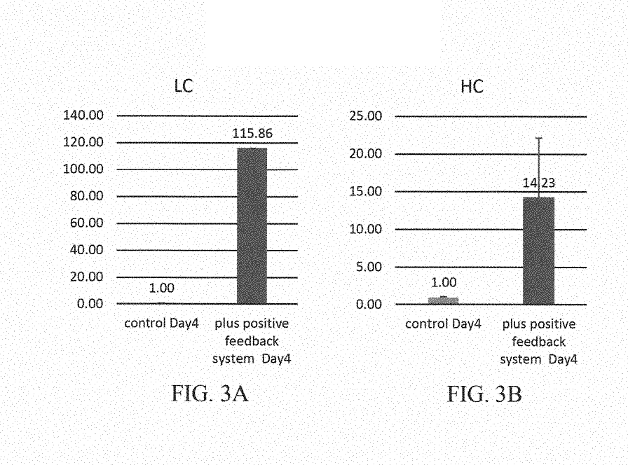

RNA was extracted from the two sets of transfected cells 4 days after transfection and quantitative polymerase chain reaction (qPCR) was performed to evaluate and compare the Trastuzumab HC/Trastuzumab LC expression levels in both sets of transfected cells. It was found that the mRNA expression level of Trastuzumab LC and Trastuzumab HC was more than 100 and 10 times higher, respectively, in cells transfected with the synthetic gene circuit system of the present invention compared to the cells transfected with pVITRO1-Trastuzumab-IgG3/κ as shown in FIG. 3a and FIG. 3b, error bars correspond to the SD.

Levels of IgG secreted into the cell culture media were assessed with an enzyme-linked immunosorbent assay (ELISA). For this comparison, pVITRO1-Trastuzumab-IgG3/κ was transiently transfected into Human Embryonic Kidney (HEK) 293 cells. The synthetic gene circuit system including four plasmids (i) CMV-tTA; (ii) TRE-tTA; and (iii) TRE-Trastuzumab HC and TRE-Trastuzumab LC was transiently transfected into a parallel population of Human Embryonic Kidney (HEK) 293 cells. Transient transfections into HEK293 cells were performed with Lipofectamine 2000 (Lifetechnologies).

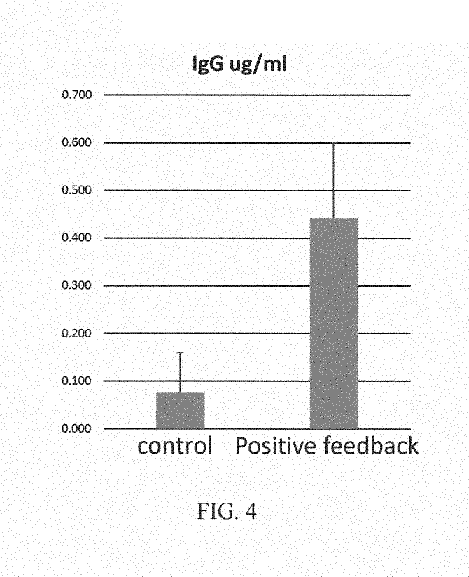

The cell culture media from both sets of transient transfections was harvested after 6 days of transfection and assayed by ELIA. It was found that the IgG expression level in the media of the cells transfected with the synthetic gene circuit system of the present invention is five times higher than the IgG expression level in the media from the pVITRO1-Trastuzumab-IgG3/κ transfected cells as shown in FIG. 4, error bars correspond to the SD.

These results show that use of a synthetic gene circuit system containing a positive feedback loop dramatically increases the production of MAB.

Sequences

SEQ ID NO: 1: tetO sequence-TCCCTATCAGTGATAGAGA
SEQ ID NO: 2: tetO sequence-CCTATCAGTGATAGA
SEQ ID NO: 3: tetO sequence-CCTGTCAGTGACAGA
SEQ ID NO: 4: tetO sequence-CCCATCAGTGATGGA
SEQ ID NO: 5: tetO sequence-CCCGTCAGTGACGGA
SEQ ID NO: 6: tetO sequence-CCTATCAGTGACGGA
SEQ ID NO: 7: encoding Tet Off Advanced 747 nucleotides
ATGTCTAGACTGGACAAGAGCAAAGTCATAAACTCTGCTCTGGAATTACTCAATGAA
GTCGGTATCGAAGGCCTGACGACAAGGAAACTCGCTCAAAAGCTGGGAGTTGAGCA
GCCTACCCTGTACTGGCACGTGAAGAACAAGCGGGCCCTGCTCGATGCCCTGGCAA
TCGAGATGCTGGACAGGCATCATACCCACTTCTGCCCCCTGGAAGGCGAGTCATGGC
AAGACTTTCTGCGGAACAACGCCAAGTCATTCCGCTGTGCTCTCCTCTCACATCGCG
ACGGGGCTAAAGTGCATCTCGGCACCCGCCCAACAGAGAAACAGTACGAAACCCTG
GAAAATCAGCTCGCGTTCCTGTGTCAGCAAGGCTTCTCCCTGGAGAACGCACTGTAC
GCTCTGTCCGCCGTGGGCCACTTTACACTGGGCTGCGTATTGGAGGATCAGGAGCAT
CAAGTAGCAAAAGAGGAAAGAGAGACACCTACCACCGATTCTATGCCCCCACTTCT
GAGACAAGCAATTGAGCTGTTCGACCATCAGGGAGCCGAACCTGCCTTCCTTTTCGG
CCTGGAACTAATCATATGTGGCCTGGAGAAACAGCTAAAGTGCGAAAGCGGCGGGC
CGGCCGACGCCCTTGACGATTTTGACTTAGACATGCTCCCAGCCGATGCCCTTGACG
ACTTTGACCTTGATATGCTGCCTGCTGACGCTCTTGACGATTTTGACCTTGACATGCT
CCCCGGGTAA
SEQ ID NO: 8: Tet Off Advanced 248 aa
MSRLDKSKVINSALELLNEVGIEGLTTRKLAQKLGVEQPTLYWHVKNKRALLDALAIE
MLDRHHTHFCPLEGESWQDFLRNNAKSFRCALLSHRDGAKVHLGTRPTEKQYETLENQ
LAFLCQQGFSLENALYALSAVGHFTLGCVLEDQEHQVAKEERETPTTDSMPPLLRQAIE
LFDHQGAEPAFLFGLELIICGLEKQLKCESGGPADALDDFDLDMLPADALDDFDLDMLP
ADALDDFDLDMLPG
SEQ ID NO: 9: encoding Tet On Advanced 751 nucleotides
ATGTCTAGACTGGACAAGAGCAAAGTCATAAACGGCGCTCTGGAATTACTCAATGG
AGTCGGTATCGAAGGCCTGACGACAAGGAAACTCGCTCAAAAGCTGGGAGTTGAGC
AGCCTACCCTGTACTGGCACGTGAAGAACAAGCGGGCCCTGCTCGATGCCCTGCCA
ATCGAGATGCTGGACAGGCATCATACCCACTTCTGCCCCCTGGAAGGCGAGTCATG
GCAAGACTTTCTGCGGAACAACGCCAAGTCATTCCGCTGTGCTCTCCTCTCACATCG
CGACGGGGCTAAAGTGCATCTCGGCACCCGCCCAACAGAGAAACAGTACGAAACCC
TGGAAAATCAGCTCGCGTTCCTGTGTCAGCAAGGCTTCTCCCTGGAGAACGCACTGT
ACGCTCTGTCCGCCGTGGGCCACTTTACACTGGGCTGCGTATTGGAGGAACAGGAGC
ATCAAGTAGCAAAAGAGGAAAGAGAGACACCTACCACCGATTCTATGCCCCCACTT
CTGAGACAAGCAATTGAGCTGTTCGACCGGCAGGGAGCCGAACCTGCCTTCCTTTTC
GGCCTGGAACTAATCATATGTGGCCTGGAGAAACAGCTAAAGTGCGAAAGCGGCGG
GCCGGCCGACGCCCTTGACGATTTTGACTTAGACATGCTCCCAGCCGATGCCCTTGA
CGACTTTGACCTTGATATGCTGCCTGCTGACGCTCTTGACGATTTTGACCTTGACATG
CTCCCCGGGTAACTAG
SEQ ID NO: 10: Tet On Advanced 248 aa
MSRLDKSKVINGALELLNGVGIEGLTTRKLAQKLGVEQPTLYWHVKNKRALLDALPIE
MLDRHHTHFCPLEGESWQDFLRNNAKSFRCALLSHRDGAKVHLGTRPTEKQYETLENQ
LAFLCQQGFSLENALYALSAVGHFTLGCVLEEQEHQVAKEERETPTTDSMPPLLRQAIEL
FDRQGAEPAFLFGLELIICGLEKQLKCESGGPADALDDFDLDMLPADALDDFDLDMLPA
DALDDFDLDMLPG
SEQ ID NO: 11: encoding TetOn 3G 747 nucleotides
ATGTCTAGACTGGACAAGAGCAAAGTCATAAACTCTGCTCTGGAATTACTCAATGGA
GTCGGTATCGAAGGCCTGACGACAAGGAAACTCGCTCAAAAGCTGGGAGTTGAGCA
GCCTACCCTGTACTGGCACGTGAAGAACAAGCGGGCCCTGCTCGATGCCCTGCCAAT
CGAGATGCTGGACAGGCATCATACCCACTCCTGCCCCCTGGAAGGCGAGTCATGGC
AAGACTTTCTGCGGAACAACGCCAAGTCATACCGCTGTGCTCTCCTCTCACATCGCG
ACGGGGCTAAAGTGCATCTCGGCACCCGCCCAACAGAGAAACAGTACGAAACCCTG
GAAAATCAGCTCGCGTTCCTGTGTCAGCAAGGCTTCTCCCTGGAGAACGCACTGTAC
GCTCTGTCCGCCGTGGGCCACTTTACACTGGGCTGCGTATTGGAGGAACAGGAGCAT
CAAGTAGCAAAAGAGGAAAGAGAGACACCTACCACCGATTCTATGCCCCCACTTCT
GAAACAAGCAATTGAGCTGTTCGACCGGCAGGGAGCCGAACCTGCCTTCCTTTTCGG
CCTGGAACTAATCATATGTGGCCTGGAGAAACAGCTAAAGTGCGAAAGCGGCGGGC
CGACCGACGCCCTTGACGATTTTGACTTAGACATGCTCCCAGCCGATGCCCTTGACG
ACTTTGACCTTGATATGCTGCCTGCTGACGCTCTTGACGATTTTGACCTTGACATGCT
CCCCGGGTAA
SEQ ID NO: 12: TetOn 3G 248 aa
MSRLDKSKVINSALELLNGVGIEGLTTRKLAQKLGVEQPTLYWHVKNKRALLDALPIEM
LDRHHTHSCPLEGESWQDFLRNNAKSYRCALLSHRDGAKVHLGTRPTEKQYETLENQL
AFLCQQGFSLENALYALSAVGHFTLGCVLEEQEHQVAKEERETPTTDSMPPLLKQAIELF
DRQGAEPAFLFGLELIICGLEKQLKCESGGPTDALDDFDLDMLPADALDDFDLDMLPAD
ALDDFDLDMLPG
SEQ ID NO: 13: encoding rtTA 747 nucleotides
ATGTCTAGACTGGACAAGAGCAAAGTCATAAACGGAGCTCTGGAATTACTCAATGG
TGTCGGTATCGAAGGCCTGACGACAAGGAAACTCGCTCAAAAGCTGGGAGTTGAGC
AGCCTACCCTGTACTGGCACGTGAAGAACAAGCGGGCCCTGCTCGATGCCCTGCCA
ATCGAGATGCTGGACAGGCATCATACCCACTTCTGCCCCCTGGAAGGCGAGTCATG
GCAAGACTTTCTGCGGAACAACGCCAAGTCATACCGCTGTGCTCTCCTCTCACATCG
CGACGGGGCTAAAGTGCATCTCGGCACCCGCCCAACAGAGAAACAGTACGAAACCC
TGGAAAATCAGCTCGCGTTCCTGTGTCAGCAAGGCTTCTCCCTGGAGAACGCACTGT
ACGCTCTGTCCGCCGTGGGCCACTTTACACTGGGCTGCGTATTGGAGGAACAGGAGC
ATCAAGTAGCAAAAGAGGAAAGAGAGACACCTACCACCGATTCTATGCCCCCACTT
CTGAGACAAGCAATTGAGCTGTTCGACCGGCAGGGAGCCGAACCTGCCTTCCTTTTC
GGCCTGGAACTAATCATATGTGGCCTGGAGAAACAGCTAAAGTGCGAAAGCGGCGG
GCCGACCGACGCCCTTGACGATTTTGACTTAGACATGCTCCCAGCCGATGCCCTTGA
CGACTTTGACCTTGATATGCTGCCTGCTGACGCTCTTGACGATTTTGACCTTGACATG
CTCCCCGGGTAA
SEQ ID NO: 14: rtTA 248 aa
MSRLDKSKVINGALELLNGVGIEGLTTRKLAQKLGVEQPTLYWHVKNKRALLDALPIE
MLDRHHTHFCPLEGESWQDFLRNNAKSYRCALLSHRDGAKVHLGTRPTEKQYETLENQ
LAFLCQQGFSLENALYALSAVGHFTLGCVLEEQEHQVAKEERETPTTDSMPPLLRQAIEL
FDRQGAEPAFLFGLELIICGLEKQLKCESGGPTDALDDFDLDMLPADALDDFDLDMLPA
DALDDFDLDMLPG
SEQ ID NO: 15: encoding tTA 1008 nucleotides
ATGTCTAGATTAGATAAAAGTAAAGTGATTAACAGCGCATTAGAGCTGCTTAATGA
GGTCGGAATCGAAGGTTTAACAACCCGTAAACTCGCCCAGAAGCTAGGTGTAGAGC
AGCCTACATTGTATTGGCATGTAAAAAATAAGCGGGCTTTGCTCGACGCCTTAGCCA
TTGAGATGTTAGATAGGCACCATACTCACTTTTGCCCTTTAGAAGGGGAAAGCTGGC
AAGATTTTTTACGTAATAACGCTAAAAGTTTTAGATGTGCTTTACTAAGTCATCGCG
ATGGAGCAAAAGTACATTTAGGTACACGGCCTACAGAAAAACAGTATGAAACTCTC
GAAAATCAATTAGCCTTTTTATGCCAACAAGGTTTTTCACTAGAGAATGCATTATAT
GCACTCAGCGCTGTGGGGCATTTTACTTTAGGTTGCGTATTGGAAGATCAAGAGCAT
CAAGTCGCTAAAGAAGAAAGGGAAACACCTACTACTGATAGTATGCCGCCATTATT
ACGACAAGCTATCGAATTATTTGATCACCAAGGTGCAGAGCCAGCCTTCTTATTCGG
CCTTGAATTGATCATATGCGGATTAGAAAAACAACTTAAATGTGAAAGTGGGTCCGC
GTACAGCCGCGCGCGTACGAAAAACAATTACGGGTCTACCATCGAGGGCCTGCTCG
ATCTCCCGGACGACGACGCCCCCGAAGAGGCGGGGCTGGCGGCTCCGCGCCTGTCC
TTTCTCCCCGCGGGACACACGCGCAGACTGTCGACGGCCCCCCCGACCGATGTCAGC
CTGGGGGACGAGCTCCACTTAGACGGCGAGGACGTGGCGATGGCGCATGCCGACGC
GCTAGACGATTTCGATCTGGACATGTTGGGGGACGGGGATTCCCCGGGTCCGGGATT
TACCCCCCACGACTCCGCCCCCTACGGCGCTCTGGATATGGCCGACTTCGAGTTTGA
GCAGATGTTTACCGATGCCCTTGGAATTGACGAGTACGGTGGGTAG
SEQ ID NO: 16: tTA 335 aa
MSRLDKSKVINSALELLNEVGIEGLTTRKLAQKLGVEQPTLYWHVKNKRALLDALAIE
MLDRHHTHFCPLEGESWQDFLRNNAKSFRCALLSHRDGAKVHLGTRPTEKQYETLENQ
LAFLCQQGFSLENALYALSAVGHFTLGCVLEDQEHQVAKEERETPTTDSMPPLLRQAIE
LFDHQGAEPAFLFGLELIICGLEKQLKCESGSAYSRARTKNNYGSTIEGLLDLPDDDAPEE
AGLAAPRLSFLPAGHTRRLSTAPPTDVSLGDELHLDGEDVAMAHADALDDFDLDMLGD
GDSPGPGFTPHDSAPYGALDMADFEFEQMFTDALGIDEYGG

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A synthetic gene circuit system, comprising:
a first expression construct comprising a nucleic acid encoding tetracycline transactivator (tTA) operably linked to a ubiquitous promoter;
a second expression construct comprising a nucleic acid encoding tetracycline transactivator (tTA) operably linked to a tetracycline responsive element (TRE);
a third expression construct comprising a nucleic acid encoding a heavy chain component of an monoclonal antibody operably linked to a TRE; and
a fourth expression construct comprising a nucleic acid encoding a light chain component of an monoclonal antibody operably linked to a TRE,
wherein expression of tTA by the first expression construct stimulates expression of tTA by the second expression construct, wherein expression of tTA by the first and second expression constructs stimulates expression of the heavy chain component of the monoclonal antibody and the light chain component of the monoclonal antibody, and wherein expression of tTA by the second expression construct establishes a positive feedback loop, thereby amplifying expression of the heavy chain component of the monoclonal antibody and the light chain component of the monoclonal antibody.

2. The system of claim 1, wherein the light chain component of the monoclonal antibody is a human light chain and the heavy chain component of the monoclonal antibody is a human heavy chain.

3. The system of claim 1, wherein the light chain component of the monoclonal antibody is a human kappa light chain or human lambda light chain.

4. The system of claim 1, wherein the heavy chain component of the monoclonal antibody is selected from the group consisting of: human gamma heavy chain (IgG), human gamma heavy chain (IgM), human gamma heavy chain (IgD), human gamma heavy chain (IgA), and human gamma heavy chain (IgE).

5. The system of claim 1, wherein the light chain component of the monoclonal antibody is a human light chain, humanized light chain, chimeric light chain, or a combination of any two or more thereof; and the heavy chain component of the monoclonal antibody is a human heavy chain, humanized heavy chain, chimeric heavy chain, or a combination of any two or more thereof.

6. The system of claim 1, wherein the light chain component of the monoclonal antibody is a fragment of a human light chain, humanized light chain, chimeric light chain, or a combination of any two or more thereof; and the heavy chain component of the monoclonal antibody is a fragment of a human heavy chain, humanized heavy chain, chimeric heavy chain, or a combination of any two or more thereof.

7. The system of claim 1, wherein the light chain component of the monoclonal antibody is a human light chain, humanized light chain, chimeric light chain, or a combination of any two or more thereof; and the heavy chain component of the monoclonal antibody is a fragment of a human heavy chain, humanized heavy chain, chimeric heavy chain, or a combination of any two or more thereof.

8. The system of claim 1, wherein one or more of the expression constructs is incorporated in a vector.

9. The system of claim 8, wherein the vector is selected from the group consisting of: plasmid, adenovirus, adeno-associated virus, retrovirus, and lentivirus.

10. The system of claim 1, wherein the constitutive promoter is cytomegalovirus (CMV) promoter.

11. A host cell comprising the system of claim 1.

12. A method of producing a monoclonal antibody, comprising:
collecting the monoclonal antibody expressed by the system present in a host cell according to claim 11.

13. The method of claim 12, wherein collecting the monoclonal antibody comprises purifying the monoclonal antibody.

14. A method of producing a monoclonal antibody, comprising:
introducing the system according to claim 1 into a host cell; and
collecting the monoclonal antibody expressed by the system.

15. The method of claim 14, wherein collecting the monoclonal antibody comprises purifying the monoclonal antibody.

16. A kit comprising the system according to claim 1.

17. The kit of claim 16, further comprising a host cell.

18. The kit of claim 17, wherein the system is present in the host cell.

19. An expression construct, comprising: a nucleic acid encoding tetracycline transactivator (tTA) operably linked to a tetracycline responsive element.
